(19) United States
(12) Patent Application Publication
(10) Pub. No.: US 2024/0161536 A1
(43) Pub. Date: May 16, 2024

(54) BIOMETRIC INFORMATION ACQUIRING APPARATUS

(71) Applicant: Sharp Semiconductor Innovation Corporation, Tenri City (JP)

(72) Inventors: DAISUKE FUNAO, Tenri City (JP); KEIICHI SHIBATA, Tenri City (JP); YUTA MOCHIZUKI, Tenri City (JP); SOTA HIDA, Tenri City (JP); KOHJI KOBAYASHI, Tenri City (JP); TOMONARI KENZAKI, Tenri City (JP)

(21) Appl. No.: 18/509,737

(22) Filed: Nov. 15, 2023

(30) Foreign Application Priority Data
Nov. 16, 2022 (JP) 2022-183544

(51) Int. Cl. G06V 40/13; G06V 40/12; G06V 40/40

(52) U.S. Cl. CPC G06V 40/1318; G06V 40/1365; G06V 40/1388; G06V 40/45

(57) ABSTRACT

A biometric information acquiring apparatus that acquires biometric information of a living body, the biometric information acquiring apparatus includes: a light-receiving element configured to receive light arriving from the living body and generate a light reception signal; and an analysis device configured to analyze the light reception signal, wherein the light-receiving element includes a first light-receiving element configured to receive first light having an intensity peak in a first wavelength band of 620 nm or more and 740 nm or less, and the light-receiving element further includes at least one of (i) a second light-receiving element configured to receive second light having an intensity peak in a second wavelength band of 370 nm or more and 600 nm or less, or (ii) a third light-receiving element configured to receive third light having an intensity peak in a third wavelength band of 760 nm or more and 1100 nm or less.

### CROSS-REFERENCE TO RELATED APPLICATION

The present application claims priority from Japanese Application 2022-183544, the content to which is hereby incorporated by reference into this application.

### BACKGROUND OF THE INVENTION

### 1. Field of the Invention

An aspect of the disclosure relates to a biometric information acquiring apparatus.

### 2. Description of the Related Art

WO 2017/104056 discloses an example of a configuration of an apparatus for acquiring biometric information (e.g., a volume pulse wave).

### SUMMARY OF THE INVENTION

There is a demand to acquire biometric information with higher accuracy.

A biometric information acquiring apparatus according to an aspect of the disclosure to meet the demand is a biometric information acquiring apparatus that acquires biometric information of a living body, the biometric information acquiring apparatus including a light-receiving element configured to receive light arriving from the living body and generate a light reception signal corresponding to the light and an analysis device configured to analyze the light reception signal to acquire the biometric information, wherein the light-receiving element includes a first light-receiving element configured to receive first light having an intensity peak in a first wavelength band of 620 nm or more and 740 nm or less and the light-receiving element further includes at least one of (i) a second light-receiving element configured to receive second light having an intensity peak in a second wavelength band of 370 nm or more and 600 nm or less or (ii) a third light-receiving element configured to receive third light having an intensity peak in a third wavelength band of 760 nm or more and 1100 nm or less.

According to an aspect of the disclosure, it is possible to acquire biometric information with higher accuracy than in the related art.

### BRIEF DESCRIPTION OF THE DRAWINGS

FIG. 1 is a block diagram illustrating a configuration of main parts of a biometric information acquiring apparatus according to a first embodiment.

FIG. 2 is a schematic front view for describing an example of a configuration of a light-receiving device according to the first embodiment.

FIG. 3 is a graph showing spectral characteristics of the light-receiving device of the first embodiment.

FIG. 4 is a schematic top view illustrating an example of an arrangement of filters of each color in the light-receiving device of the first embodiment.

FIG. 5 is a diagram for describing a modification of the biometric information acquiring apparatus of the first embodiment.

FIG. 6 is a block diagram illustrating a configuration of main parts of a biometric information acquiring apparatus according to a second embodiment.

FIG. 7 is a diagram illustrating an example of a configuration of a light-emitting device and a light-receiving device in the biometric information acquiring apparatus of the second embodiment.

FIG. 8 is a graph showing a wavelength spectrum of test light emitted from the light-emitting device of the second embodiment.

FIG. 9 is a diagram for describing a modification of the biometric information acquiring apparatus of the second embodiment.

### DETAILED DESCRIPTION OF THE INVENTION

### First Embodiment

A first embodiment will be described below. In each of the subsequent embodiments, components having the same functions as components described in the first embodiment are given the same reference numerals for convenience of explanation and description thereof will not be repeated. Description of known technical matters will be omitted as appropriate for the sake of brevity. The components and numerical values described in this specification are all merely examples unless otherwise contradicted. Thus, for example, the positional relationships of the components are not limited to the examples illustrated in the drawings unless otherwise contradicted. The drawings are not necessarily drawn to scale.

### Example of Configuration of biometric information Acquiring Apparatus 1

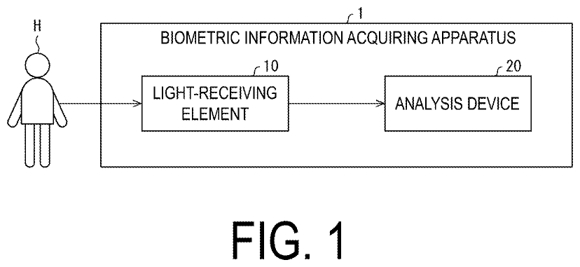

FIG. 1 is a block diagram illustrating a configuration of main parts of a biometric information acquiring apparatus 1 of the first embodiment. The biometric information acquiring apparatus 1 acquires biometric information of a living body H. The living body H is not particularly limited as long as it is a living object whose biometric information can be acquired by the biometric information acquiring apparatus 1. In FIG. 1, a human being is illustrated as the living body H. The biometric information acquiring apparatus 1 includes a light-receiving device 10 and an analysis device 20. In an example, the analysis device 20 may be a control device responsible for overall control of the components of the biometric information acquiring apparatus 1.

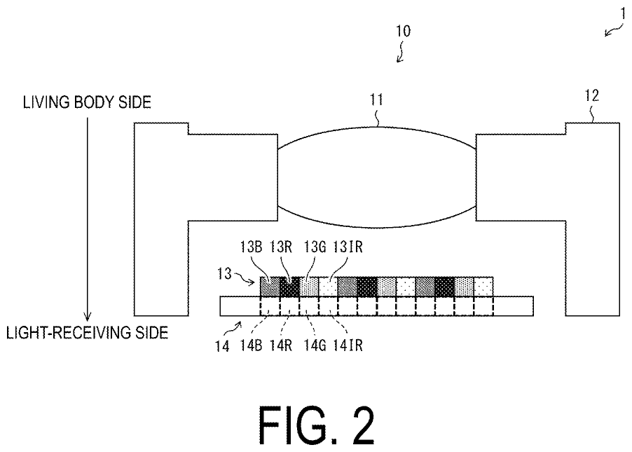

The light-receiving device 10 includes a light-receiving element (e.g., a light-receiving element 14 in FIG. 2 described below) that receives light arriving from the living body H and generates a light reception signal corresponding to the received light. The light-receiving element is also referred to as a photoelectric conversion device. In the example of the first embodiment, it is assumed that the space where the living body H is present is illuminated by ambient light. It is also assumed that the ambient light is white light. Part of the ambient light is reflected by the living body H (more specifically, the skin of the living body H). Then, the ambient light reflected by the living body H is incident on the light-receiving device 10.

The analysis device 20 analyzes the light reception signal to acquire (e.g., derive) biometric information of the living body H. The case where the analysis device 20 acquires a volume pulse wave of a living body H as biometric information will be mainly illustrated in the first embodiment.

FIG. 2 is a schematic front view for describing an example of a configuration of the light-receiving device 10. The light-receiving device 10 includes a light guide member 11, a support member 12, a spectral filter 13, and a light-receiving element 14. The lower side of the page of FIG. 2 is the light-receiving side (the side where the light-receiving element 14 is located) and the upper side thereof is the living body side.

In the example of FIG. 2, the light-receiving device 10 includes the light-receiving element 14, the spectral filter 13, and the light guide member 11 in this order from the bottom to the top. The light guide member 11 guides the light incident on the light-receiving device 10 to the light-receiving side. In FIG. 2, a lens is illustrated as the light guide member 11. The support member 12 supports the light guide member 11. The support member 12 in the example of FIG. 2 is a lens holder.

The spectral filter 13 transmits light having an intensity peak in a predetermined wavelength band. The spectral filter 13 is also referred to as a color filter or an optical spectral filter. The spectral filter 13 is located to cover at least a part of the light-receiving element 14 when viewed from the living body side. Thus, white light which is the ambient light is separated by the spectral filter 13 and the separated light is incident on the light-receiving element 14.

The light-receiving element 14 in the example of the first embodiment is a solid-state imaging element. The solid-state imaging element may be a CMOS image sensor (CIS) or a charge coupled device (CCD). The solid-state imaging element can be used as a light-receiving element to capture an image showing a predetermined part of the living body H (e.g., a face image of the living body H). The analysis device 20 can analyze the image to derive more diverse biometric information.

In this specification, a wavelength band of 620 nm or more and 740 nm or less is referred to as a first wavelength band, a wavelength band of 370 nm or more and 600 nm or less is referred to as a second wavelength band, and a wavelength band of 760 nm or more and 1100 nm or less is referred to as a third wavelength band. Light having an intensity peak in the first wavelength band is referred to as first light, light having an intensity peak in the second wavelength band is referred to as second light, and light having an intensity peak in the third wavelength band is referred to as third light.

Also, in this specification, a range of 370 nm or more and 490 nm or less in the second wavelength band is referred to as a first type of second wavelength band and a range of more than 490 nm and 590 nm or less is referred to as a second type of second wavelength band. Light having an intensity peak in the first type of second wavelength band is referred to as a first type of second light and light having an intensity peak in the second type of second wavelength band is referred to as a second type of second light.

As is apparent to those skilled in the art, the wavelength band of red (R) light belongs to the first wavelength band, such that red light is an example of the first light. The wavelength band of infrared (IR) light belongs to the third wavelength band, such that infrared light is an example of the third light. The wavelength band of blue (B) light belongs to the first type of second wavelength band and the wavelength band of green (G) light belongs to the second type of second wavelength band, such that blue light is an example of the first type of second light and green light is an example of the second type of second light. Accordingly, the second light may be, for example, blue light or green light.

The spectral filter 13 in the example of FIG. 2 includes red light filters 13R, blue light filters 13B, green light filters 13G, and infrared light filters 13IR. In this specification, a spectral filter that transmits the first light is referred to as a first filter, a spectral filter that transmits the second light is referred to as a second filter, and a spectral filter that transmits the third light is referred to as a third filter. The red light filters 13R are examples of a first filter. Both the blue light filters 13B and the green light filters 13G are examples of a second filter. The infrared light filters 13IR are examples of a third filter.

Also, in this specification, a spectral filter that transmits the first type of second light is referred to as a first type of second filter and a spectral filter that transmits the second type of second light is referred to as a second type of second filter. The blue light filters 13B are examples of a first type of second filter and the green light filters 13G are examples of a second type of second filter.

The first filter (e.g., each red light filter 13R) ideally has spectral characteristics that selectively transmits only the first light (e.g., red light). Thus, the first filter has high light transmittance (ideally, light transmittance of 100%) in the first wavelength band (e.g., the wavelength band of red light) and has low light transmittance (ideally, light transmittance of 0%) in wavelength bands excluding the first wavelength band. This description regarding the light transmittance of the first filter applies similarly to spectral filters corresponding to other wavelength bands.

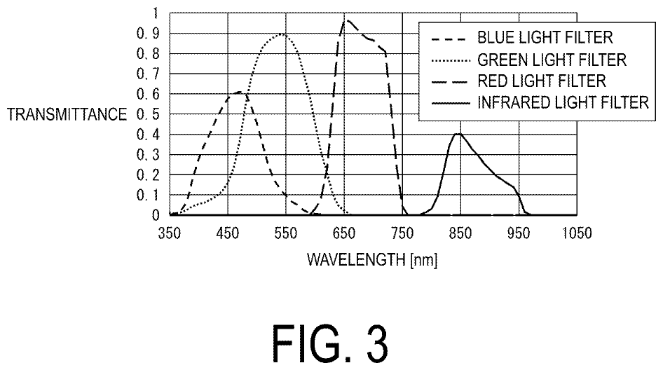

FIG. 3 is a graph showing spectral characteristics of the light-receiving device 10 in FIG. 2 (specifically, the spectral filter 13 in FIG. 2). In FIG. 3, the horizontal axis represents the wavelength of light and the vertical axis represents the transmittance of light (light transmittance). As described above, the spectral filter 13 of FIG. 2 includes the red light filters 13R, the blue light filters 13B, the green light filters 13G, and the infrared light filters 13IR. This configuration realizes the spectral characteristics shown in FIG. 3. According to the spectral characteristics, white light incident on the light-receiving device 10 is separated into red light (first light), blue light (a first type of second light), green light (a second type of second light), and infrared light (third light).

As shown in FIG. 3, each blue light filter 13B has a light transmittance peak at or near a wavelength of 470 nm. Thus, blue light separated by the blue light filter 13B has an intensity peak at or near the wavelength of 470 nm. Each green light filter 13G has a light transmittance peak at or near a wavelength of 540 nm. Thus, green light separated by the green light filter 13G has an intensity peak at or near the wavelength of 540 nm. Each red light filter 13R has a light transmittance peak at or near a wavelength of 650 nm. Thus, red light separated by the red light filter 13R has an intensity peak at or near the wavelength of 650 nm. Each infrared light filter 13IR has a light transmittance peak at or near a wavelength of 850 nm. Thus, infrared light separated by the infrared light filter 13IR has an intensity peak at or near the wavelength of 850 nm.

Reference is made back to FIG. 2. The light-receiving element 14 in the example of FIG. 2 includes red light-receiving elements 14R, blue light-receiving elements 14B, green light-receiving elements 14G, and infrared light-receiving elements 14IR. In this specification, a light-receiving element that receives the first light is referred to as a first light-receiving element, a light-receiving element that receives the second light is referred to as a second light-receiving element, and a light-receiving element that receives the third light is referred to as a third light-receiving element. The red light-receiving elements 14R are examples of a first light-receiving element. Both the blue light-receiving elements 14B and the green light-receiving elements 14G are examples of a second light-receiving element. The infrared light-receiving elements 14IR are examples of a third light-receiving element.

Also, in this specification, a light-receiving element that receives the first type of second light is referred to as a first type of second light-receiving element and a light-receiving element that receives the second type of second light is referred to as a second type of second light-receiving element. The blue light-receiving elements 14B are examples of a first type of second light-receiving element and the green light-receiving elements 14G are examples of a second type of second light-receiving element.

As illustrated in FIG. 2, the blue light filters 13B cover the blue light-receiving elements 14B when viewed from the living body side. Thus, the blue light-receiving elements 14B receive the blue light separated by the blue light filters 13B. The green light filters 13G cover the green light-receiving elements 14G when viewed from the living body side. Thus, the green light-receiving elements 14G receive the green light separated by the green light filters 13G. The red light filters 13R cover the red light-receiving elements 14R when viewed from the living body side. Thus, the red light-receiving elements 14R receive the red light separated by the red light filters 13R. The infrared light filters 13IR cover the infrared light-receiving element 14IR when viewed from the living body side. Thus, the infrared light-receiving element 14IR receive the infrared light separated by the infrared light filters 13IR.

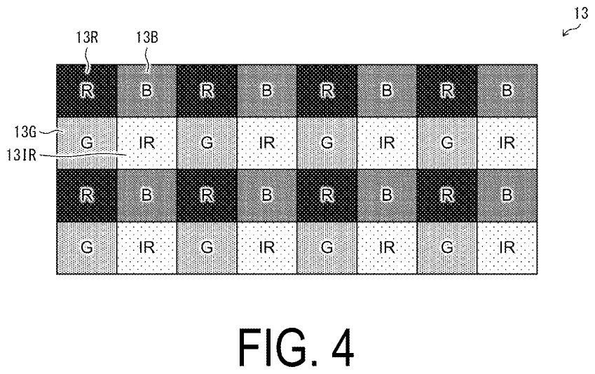

FIG. 4 is a schematic top view illustrating an example of an arrangement of filters of each color in the light-receiving device 10 of FIG. 2. As illustrated in FIG. 4, filters of each color may be regularly arranged two-dimensionally. Thus, the arrangement of filters of each color may be a two-dimensional array. Accordingly, the arrangement of light-receiving elements of each color may also be a two-dimensional array corresponding to the arrangement of filters of each color.

As is apparent from the above description, the first light-receiving elements may be realized as regions covered by first filters in a light-receiving region of a bulk solid-state imaging element. The first light-receiving elements each generate a first light reception signal corresponding to the received first light. Thus, for example, the red light-receiving elements 14R each generate a light reception signal of red light corresponding to the received red light. This description regarding the first light-receiving elements applies similarly to light-receiving elements corresponding to other wavelength bands.

The analysis device 20 acquires the first to third light reception signals from the first to third light-receiving elements, respectively. In an example, the analysis device 20 can acquire a volume pulse wave of the living body H by analyzing time series data of the first to third light reception signals (e.g., performing signal processing on time series data of the first to third light reception signals).

Incidentally, signal components of a volume pulse wave (hereinafter referred to as volume pulse wave components) included in each light reception signal are relatively small and noise may also be superimposed on each light reception signal. Noise is caused, for example, by fluctuations in the amount of ambient light and body movements of the living body H. Thus, the signal processing may include arbitrary numerical processing for noise removal. Examples of numerical processing include averaging, calculation using a median, logarithmization, and scaling. Performing noise removal can acquire a volume pulse wave of the living body H with higher accuracy.

### Effects of Biometric information Acquiring Apparatus 1

The technique itself for detecting biometric information (e.g., a volume pulse wave) using a solid-state imaging element as a light-receiving element is well known. However, general solid-state imaging elements are designed for the purpose of capturing images of subjects. Human eyes have high visual sensitivity in the green wavelength band. Hemoglobin contained in the skin of a living body has a particularly high light absorption rate at a wavelength of 550 nm belonging to the green wavelength band. Thus, in the related art, general solid-state imaging elements have been used without modification to detect a volume pulse wave.

For example, the technique disclosed in WO 2017/104056 acquires a light reception signal of blue light, a light reception signal of green light, and a light reception signal of red light using a general solid-state imaging element. The technique disclosed in WO 2017/104056 uses the light reception signal of red light or the light reception signal of blue light as a reference signal for the light reception signal of green light. Specifically, the technique disclosed in WO 2017/104056 uses the reference signal to perform signal processing for removing noise from the light reception signal of green light which is a signal of interest.

A general solid-state imaging element is designed to receive red light having an intensity peak in a wavelength band around 600 nm and blue light having an intensity peak in a wavelength band around 450 nm. However, in these wavelength bands, the light reception signal of red light or the light reception signal of blue light includes volume pulse wave components that are large to some extent. Therefore, signal processing of the technique of WO 2017/104056 reduces volume pulse wave components included in the signal of interest as it removes noise using a reference signal.

The inventors of the present application (hereinafter abbreviated as “inventors”) have newly devised a biometric information acquiring apparatus according to an aspect of the disclosure (e.g., the biometric information acquiring apparatus 1) in view of the above problems of the technique of WO 2017/104056. The inventors have discovered through experiments that the light absorption rate of hemoglobin is particularly small in the first wavelength band described above. Namely, the inventors have discovered that volume pulse wave components included in a first light reception signal are particularly small in the first wavelength band. Based on this new knowledge, the inventors have further discovered a new idea of using a first light reception signal as a reference signal.

Therefore, a biometric information acquiring apparatus according to an aspect of the disclosure includes a first light-receiving element. Thus, a first light reception signal can be acquired as a reference signal. Use of the first signal as a reference signal can minimize the decrease in volume pulse wave components of a signal of interest due to noise removal. Thus, the biometric information acquiring apparatus according to the aspect of the disclosure can acquire a volume pulse wave with higher accuracy than the technique of WO 2017/104056.

A signal of interest in a biometric information acquiring apparatus according to an aspect of the disclosure may be either a second light reception signal or a third light reception signal. Thus, the biometric information acquiring apparatus according to the aspect of the disclosure only needs to further include at least one of a second light-receiving element that receives second light or a third light-receiving element that receives third light. As is apparent from the above description, the biometric information acquiring apparatus 1 in the example of FIG. 2 includes both second light-receiving elements and third light-receiving elements.

A biometric information acquiring apparatus according to an aspect of the disclosure only needs to include at least one of a first type of second light-receiving element or a second type of second light-receiving element as a second light-receiving element. As is apparent from the above description, the biometric information acquiring apparatus 1 in the example of FIG. 2 includes both a first type of second light-receiving elements and a second type of second light-receiving elements. Providing more diverse types of light-receiving elements can remove noise from the signal of interest using more diverse methods.

Incidentally, human skin contains melanin. However, it is difficult to identify the wavelength band of noise derived from melanin in advance because there are large individual differences in the content of melanin in the skin. However, by providing light-receiving elements of diverse types as described above, the biometric information acquiring apparatus according to the aspect of the disclosure can reduce noise derived from melanin and can also reduce noise derived from fluctuations in the amount of ambient light, body movements of the living body, and the like. Therefore, the biometric information acquiring apparatus according to the aspect of the disclosure can acquire a volume pulse wave with higher accuracy than in the related art.

As is apparent to those skilled in the art, a biometric information acquiring apparatus according to an aspect of the disclosure can further derive other biometric information based on a volume pulse wave. For example, the biometric information acquiring apparatus can acquire the heart rate, blood pressure, stress level, or the like of the living body based on the volume pulse wave. As described above, a biometric information acquiring apparatus according to an aspect of the disclosure can acquire such biometric information with higher accuracy than in the related art.

Incidentally, a typical example of hemoglobin contained in the skin of a living body is oxyhemoglobin. It is known that the light absorption rate of oxyhemoglobin increases as the wavelength of light becomes shorter. Therefore, spectral characteristics of a biometric information acquiring apparatus according to an aspect of the disclosure may be set based on the light absorption characteristics of oxyhemoglobin. A wavelength spectrum of test light described in a second embodiment that will be described later may also be set based on the light absorption characteristics.

The first embodiment illustrates the case where the biometric information acquiring apparatus includes a spectral filter. The spectral filter only needs to be designed to correspond to a light-receiving element. Thus, the spectral filter only needs to include a first filter and further include at least one of a second filter or a third filter. The biometric information acquiring apparatus 1 in the example of FIG. 2 includes both second filters and third filters.

A biometric information acquiring apparatus according to an aspect of the disclosure only needs to include at least one of a first type of second filter or a second type of second filter as a second filter. The biometric information acquiring apparatus 1 in the example of FIG. 2 includes both a first type of second filters and a second type of second filters.

### Modified Example

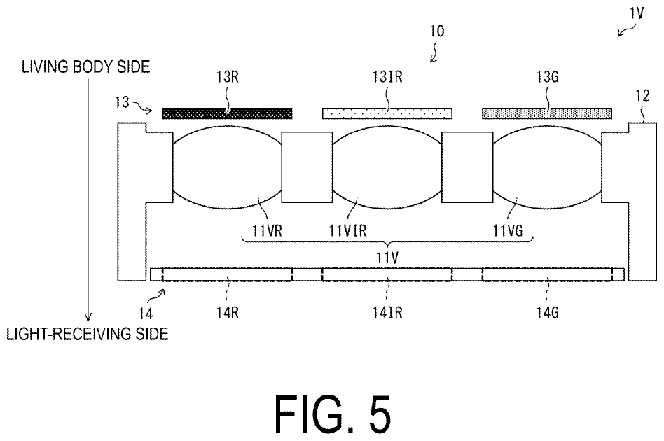

FIG. 5 is a diagram for describing a modification of the biometric information acquiring apparatus of the first embodiment. FIG. 5 is a diagram corresponding to FIG. 2. The biometric information acquiring apparatus in FIG. 5 is referred to as a biometric information acquiring apparatus 1V for convenience of explanation.

A light-receiving device 10 in the example of FIG. 5 includes a light guide member 11V instead of the light guide member 11. A spectral filter 13 in the example of FIG. 5 is located above the light guide member 11V. Thus, the positional relationship between the spectral filter 13 and the light guide member is not limited to the example illustrated in FIG. 2 described above. As is apparent from the second embodiment that will be described later, a light-receiving device according to an aspect of the disclosure may not include a light guide member.

Unlike the example in FIG. 2, the spectral filter 13 may not include a blue light filter 13B (a first type of second filter) as illustrated in FIG. 5. Thus, unlike the example of FIG. 2, the light-receiving element 14 may not include a blue light-receiving element 14B (a first type of second light-receiving element) as illustrated in FIG. 5.

However, it is to be noted that the configuration of FIG. 5 is merely an example as is apparent to those skilled in the art. Thus, for example, the light-receiving element 14 in the example of FIG. 5 may include a blue light-receiving element 14B instead of a green light-receiving element 14G (a second type of second light-receiving element). In this case, the spectral filter 13 in the example of FIG. 5 may include a blue light filter 13B instead of a green light filter 13G.

In another example, the light-receiving element 14 in the example of FIG. 5 may include both a blue light-receiving element 14B and a green light-receiving element 14G as second light-receiving elements. In this case, the spectral filter 13 in the example of FIG. 5 may include both a blue light filter 13B and a green light filter 13G.

When the light-receiving element 14 includes a blue light-receiving element 14B as a second light-receiving element, it becomes easier to identify the color of the skin of the living body in an image captured by the light-receiving element 14. Incidentally, there may be individual differences in the magnitudes of volume pulse wave components included in each of a light reception signal of blue light and a light reception signal of green light. For example, it is known that volume pulse wave components included in a light reception signal of blue light are greater than volume pulse wave components included in a light reception signal of green light for some people. When the light-receiving element 14 includes a blue light-receiving element 14B as a second light-receiving element, such biometric information of people can be acquired with high accuracy.

The light guide member 11V in the example of FIG. 5 includes a first light guide member 11VR, a second light guide member 11VG, and a third light guide member 11VIR. Thus, in the example of FIG. 5, a separate light guide member is provided for each color, unlike the example of FIG. 2.

The first light guide member 11VR is located below a red light filter 13R (a first filter). The first light guide member 11VR guides red light (first light) separated by the red light filter 13R to a red light-receiving element 14R (a first light-receiving element). The second light guide member 11VG guides green light (a second type of second light) separated by the green light filter 13G (a second type of second filter) to a green light-receiving element 14G (a first type of second light-receiving element). The third light guide member 11VIR guides infrared light (third light) separated by an infrared light filter 13IR (a third filter) to an infrared light-receiving element 14IR (a third light-receiving element).

According to the configuration of FIG. 5, the light guide members can be made smaller compared to the configuration of FIG. 2. Thus, for example, the light-receiving device can be made thinner. The light-receiving device can also be made lighter. The spectral filter may not be in contact with the light-receiving element as illustrated in FIG. 5. According to the configuration of FIG. 5, the spectral filter can be more easily disposed within the light-receiving device than in the configuration of FIG. 2.

### Second Embodiment

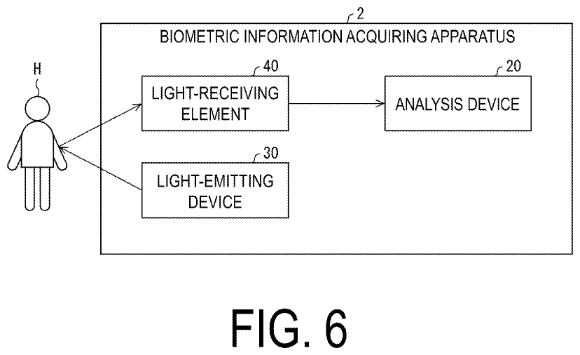

FIG. 6 is a block diagram illustrating a configuration of main parts of a biometric information acquiring apparatus 2 according to the second embodiment. Unlike the biometric information acquiring apparatus 1, the biometric information acquiring apparatus 2 further includes a light-emitting device 30. The biometric information acquiring apparatus 2 includes a light-receiving device 40 instead of the light-receiving device 10 of the first embodiment.

As illustrated in FIG. 6, the light-emitting device 30 emits test light toward a living body H. The light-receiving device 40 includes a light-receiving element 44 described below as a light-receiving element. The light-receiving element 44 receives the test light reflected by the living body H. Thus, the light-receiving element 44 generates a light reception signal corresponding to the test light.

The light-receiving element 44 in the example of the second embodiment is a photodiode. Thus, a light-receiving element according to an aspect of the disclosure may not be a solid-state imaging element. That is, a light-receiving device according to an aspect of the disclosure may not have the function of capturing an image of the living body H. Use of a photodiode as a light-receiving element can reduce the manufacturing cost of the biometric information acquiring apparatus.

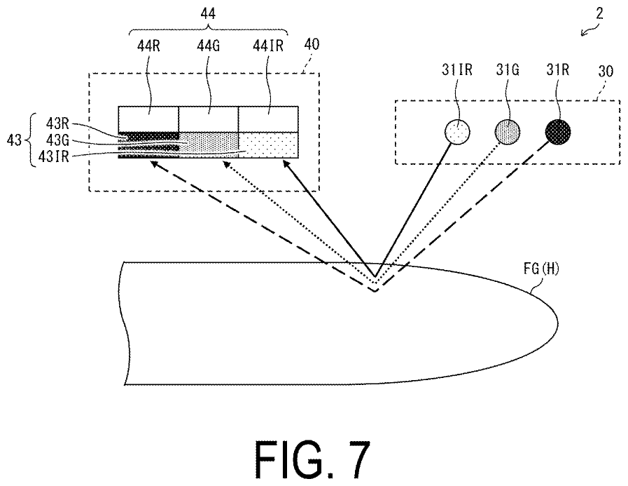

FIG. 7 is a diagram illustrating an example of a configuration of the light-emitting device 30 and the light-receiving device 40 in the biometric information acquiring apparatus 2. The light-emitting device 30 emits test light toward a predetermined part of the living body H. The predetermined part is not particularly limited as long as it is a part from which biometric information according to an aspect of the disclosure (e.g., a volume pulse wave) can be acquired. In the example of FIG. 7, a finger FG of the living body H is illustrated as the predetermined part.

The light-emitting device 30 in the example of FIG. 7 includes a red light source 31R that emits red light toward the living body H as test light, a green light source 31G that emits green light toward the living body H as test light, and an infrared light source 31IR that emits infrared light toward the living body H as test light.

In this specification, a light source that emits first light toward a living body as test light is referred to as a first light source, a light source that emits second light toward a living body as test light is referred to as a second light source, and a light source that emits third light toward a living body as test light is referred to as a third light source. The red light source 31R is an example of the first light source. The green light source 31G is an example of the second light source, and the infrared light source 31IR is an example of the third light source.

As is apparent from the description regarding light-receiving elements in the first embodiment, a light-emitting device according to an aspect of the disclosure only needs to include the first light source and at least one of the second light source or the third light source. A light-emitting device according to an aspect of the disclosure may not include arbitrary one of the first to third light sources as will be described later.

In this specification, a light source that emits the first type of second light toward a living body as test light is referred to as a first type of second light source and a light source that emits the second type of second light toward a living body as test light is referred to as a second type of second light source. The green light source 31G is an example of the second type of second light source. A light-emitting device according to an aspect of the disclosure only needs to include at least one of the first type of second light source or the green light source 31G as a second light source as is apparent from the correspondence with the description regarding light-receiving elements in the first embodiment. Thus, the light-emitting device 30 may further include a blue light source that emits blue light toward the living body H as test light. The blue light source is an example of the first type of second light source.

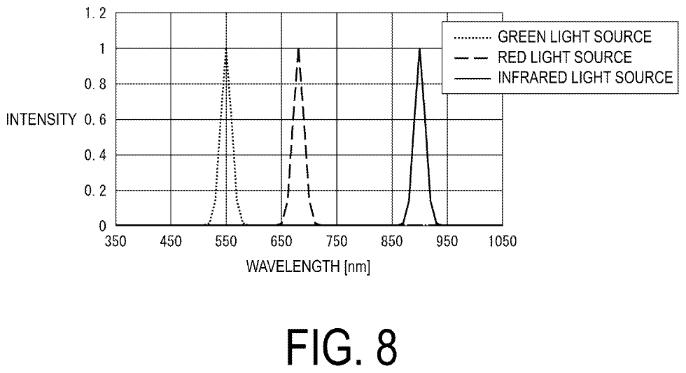

FIG. 8 is a graph showing a wavelength spectrum of test light emitted from the light-emitting device 30 of FIG. 7. In FIG. 8, the horizontal axis represents the wavelength of light and the vertical axis represents the intensity of light. The vertical axis in FIG. 8 is normalized. As described above, the light-emitting device 30 in FIG. 7 includes the red light source 31R, the green light source 31G, and the infrared light source 31IR. This configuration realizes the spectrum shown in FIG. 8. As shown in FIG. 8, green light emitted from the green light source 31G has an intensity peak at or near a wavelength of 550 nm. Red light emitted from the red light source 31R has an intensity peak at or near a wavelength of 680 nm. Infrared light emitted from the infrared light source 31IR has an intensity peak at or near a wavelength of 900 nm.

Referring again to FIG. 7, the light-receiving device 40 will be described. The light-receiving device 40 in the example illustrated in FIG. 7 includes a spectral filter 43 and a light-receiving element 44. The spectral filter 43 in the example of FIG. 7 includes a red light filter 43R, a green light filter 43G, and an infrared light filter 43IR. The red light filter 43R is an example of a first filter. The green light filter 43G is an example of a second filter (more specifically, an example of a second type of second filter). The infrared light filter 43IR is an example of a third filter.

The light-receiving element 44 in the example of FIG. 7 includes a red light-receiving element 44R, a green light-receiving element 44G, and an infrared light-receiving element 44IR. The red light-receiving element 44R is an example of a first light-receiving element. The green light-receiving element 44G is an example of a second light-receiving element (more specifically, an example of a second type of second light-receiving element). The infrared light-receiving element 44IR is an example of a third light-receiving element.

When the light-emitting device 30 further includes a first type of second light source, the light-receiving element 44 may further include a first type of second light-receiving element (e.g., a blue light-receiving element). In this case, the spectral filter 43 may further include a first type of second filter (e.g., a blue light filter).

In the example of FIG. 7, the red light source 31R, the green light source 31G, and the infrared light source 31IR are compactly arranged in the light-emitting device 30. Accordingly, the red light-receiving element 44R, the green light-receiving element 44G, and the infrared light-receiving element 44IR are also compactly arranged in the light-receiving device 40. The red light source 31R, the green light source 31G, and the infrared light source 31IR each emit test light toward a common position on the finger FG. Thus, in the example of FIG. 7, the common position is irradiated with mixed light in which red light, green light, and infrared light are mixed. The mixed light is reflected at the common position and then directed to the light-receiving device 40. The mixed light is separated by the spectral filter 43 and then incident on the light-receiving element 44.

As illustrated in FIG. 7, the red light filter 43R covers the red light-receiving element 44R when viewed from the living body side. Thus, the red light-receiving element 44R receives red light separated by the red light filter 13R. The green light filter 43G covers the green light-receiving element 44G when viewed from the living body side. Thus, the green light-receiving element 44G receives green light separated by the green light filter 43G. The infrared light filter 43IR covers the infrared light-receiving element 44IR when viewed from the living body side. Thus, the infrared light-receiving element 44IR receives infrared light separated by the infrared light filter 43IR.

The analysis device 20 acquires a light reception signal of red light (a first light reception signal) from the red light-receiving element 44R, a light reception signal of green light (a second light reception signal) from the green light-receiving element 44G, and a light reception signal of infrared light (a third light reception signal) from the infrared light-receiving element 44IR. The analysis device 20 analyzes the first to third light reception signals to acquire biometric information of the living body H.

The biometric information acquiring apparatus 2 differs from the biometric information acquiring apparatus 1 in that it includes a light source. Accordingly, the biometric information acquiring apparatus 2 can acquire biometric information of the living body H, for example, even when the amount of ambient light is small. Thus, the second embodiment can provide a biometric information acquiring apparatus that is more convenient for the user.

In an example, the biometric information acquiring apparatus 2 may be realized as a portable device. The biometric information acquiring apparatus 2 may also be realized as a contact-type device. When the biometric information acquiring apparatus 2 is a contact-type device, the light-emitting device 30 and the light-receiving device 40 can be brought sufficiently close to (e.g., brought into contact with) the finger FG. Therefore, it is possible to reduce the possibility that noise derived from ambient light will be included in the light reception signal. As a result, it is possible to increase the accuracy of biometric information acquired by the biometric information acquiring apparatus 2.

### Another Example of Configuration of Light-Emitting Device 30

The light-emitting device 30 may include a white light source that emits white light toward the living body H as test light. This is because the spectral filter 43 in the example of FIG. 7 can separate white light into red light, green light, and infrared light. Thus, the light-emitting device 30 may not include separate light sources that emit test light having different peak wavelengths, respectively.

### Modified Example

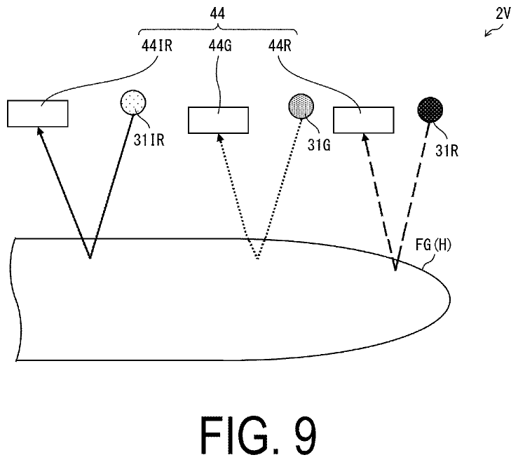

FIG. 9 is a diagram for describing a modification of the biometric information acquiring apparatus 2. For convenience of explanation, the biometric information acquiring apparatus in FIG. 9 is referred to as a biometric information acquiring apparatus 2V. FIG. 9 is a diagram corresponding to FIG. 7.

In the example of FIG. 9, unlike the example of FIG. 7, a red light source 31R, a green light source 31G, and an infrared light source 31IR are arranged apart from each other. A red light-receiving element 44R, a green light-receiving element 44G, and an infrared light-receiving element 44IR are also arranged apart from each other. Also, the biometric information acquiring apparatus 2V does not include the spectral filter 43, unlike the biometric information acquiring apparatus 2.

The red light source 31R, the green light source 31G, and the infrared light source 31IR in the example of FIG. 9 emit test light toward different positions of the finger FG, respectively. Thus, in the example of FIG. 9, the light-emitting device 30 does not irradiate the finger FG with mixed light, unlike the example of FIG. 7.

The red light source 31R emits red light toward a first position on the finger FG (e.g., the tip of the finger FG). The red light-receiving element 44R is arranged to receive the red light reflected at the first position. The green light source 31G emits green light toward a second position on the finger FG (e.g., the center of the finger FG). The green light-receiving element 44G is arranged to receive the green light reflected at the second position. The infrared light source 31IR emits infrared light toward a third position on the finger FG (e.g., the base of the finger FG). The infrared light-receiving element 44IR is arranged to receive the infrared light reflected at the third position.

According to the arrangement of the red light source 31R, the green light source 31G, and the infrared light source 31IR and the red light-receiving element 44R, the green light-receiving element 44G, and the infrared light-receiving element 44IR in FIG. 9, green light reflected at the second position and infrared light reflected at the third position are hardly incident on the red light-receiving element 44R. This allows the red light-receiving element 44R to receive red light without requiring the red light filter 43R.

Similarly, according to the arrangement illustrated in FIG. 9, red light reflected at the first position and infrared light reflected at the third position are hardly incident on the green light-receiving element 44G. This allows the green light-receiving element 44G to receive green light without requiring the green light filter 43G. Moreover, according to this arrangement, red light reflected at the first position and green light reflected at the second position are hardly incident on the infrared light-receiving element 44IR. This allows the infrared light-receiving element 44IR to receive infrared light without requiring the infrared light filter 43IR. A biometric information acquiring apparatus according to an aspect of the disclosure may not include a spectral filter as described above.

### Implementation Example by Software

The functions of each of the biometric information acquiring apparatuses 1 to 2V (hereinafter referred to as an “apparatus”) are implemented by a program for causing a computer to function as the apparatus, the program causing the computer to function as each control block of the apparatus (particularly the analysis device 20).

In this case, the apparatus includes a computer including at least one control device (e.g., processor) and at least one storage device (e.g., memory) as hardware components for executing the program. By executing the program with the control device and the storage device, each function described in the above-described each embodiment is achieved.

The program may be stored in one or a plurality of computer-readable non-transitory storage medium. The storage medium may or may not be included in the device described above. In the latter case, the program may be supplied to the device via any transmission medium, wired or wireless.

Furthermore, some or all of the functions of each control block can be implemented by a logic circuit. For example, an integrated circuit in which a logic circuit functioning as each of the control blocks described above is formed is also included in the scope of an aspect of the disclosure. In addition to this, it is also possible to realize the function of each control block described above by, for example, a quantum computer.

Each process described in each of the above embodiments may be executed by artificial intelligence (AI). In this case, the AI may operate on the control device or may operate on another device (e.g., an edge computer or a cloud server).

### Supplement

A biometric information acquiring apparatus according to a first aspect of the disclosure is a biometric information acquiring apparatus that acquires biometric information of a living body, the biometric information acquiring apparatus including a light-receiving element configured to receive light arriving from the living body and generate a light reception signal corresponding to the light and an analysis device configured to analyze the light reception signal to acquire the biometric information, wherein the light-receiving element includes a first light-receiving element configured to receive first light having an intensity peak in a first wavelength band of 620 nm or more and 740 nm or less, and the light-receiving element further includes at least one of (i) a second light-receiving element configured to receive second light having an intensity peak in a second wavelength band of 370 nm or more and 600 nm or less or (ii) a third light-receiving element configured to receive third light having an intensity peak in a third wavelength band of 760 nm or more and 1100 nm or less.

In a biometric information acquiring apparatus according to a second aspect of the disclosure, in the first aspect, the light-receiving element may include both the second light-receiving element and the third light-receiving element.

In a biometric information acquiring apparatus according to a third aspect of the disclosure, in the second aspect, the second light-receiving element may include at least one of (i) a first type of second light-receiving element configured to receive a first type of second light having an intensity peak in a first type of second wavelength band of 370 nm or more and 490 nm or less or (ii) a second type of second light-receiving element configured to receive a second type of second light having an intensity peak in a second type of second wavelength band of more than 490 nm and 590 nm or less.

In a biometric information acquiring apparatus according to a fourth aspect of the disclosure, in the third aspect, the first light may be red light, the first type of second light may be blue light, the second type of second light may be green light, and the third light may be infrared light.

In a biometric information acquiring apparatus according to a fifth aspect of the disclosure, in any one of the first to fourth aspects, the biometric information acquiring apparatus may further include a spectral filter including a first filter configured to transmit the first light, wherein the spectral filter may further include at least one of (i) a second filter configured to transmit the second light or (ii) a third filter configured to transmit the third light.

In a biometric information acquiring apparatus according to a sixth aspect of the disclosure, in any one the first to fifth aspects, the biometric information acquiring apparatus may further include a light-emitting device configured to emit test light toward the living body, wherein the light-receiving element may be configured to receive the test light reflected by the living body.

In a biometric information acquiring apparatus according to a seventh aspect of the disclosure, in the sixth aspect, the light-emitting device may include a first light source configured to emit the first light toward the living body as the test light and the light-emitting device may further include at least one of (i) a second light source configured to emit the second light toward the living body as the test light or (ii) a third light source configured to emit the third light toward the living body as the test light.

In a biometric information acquiring apparatus according to an eighth aspect of the disclosure, in the sixth aspect, the light-emitting device may include a white light source configured to emit white light toward the living body as the test light.

In a biometric information acquiring apparatus according to a ninth aspect of the disclosure, in any one of the first to eighth aspects, the light-receiving element may be a solid-state imaging element.

In a biometric information acquiring apparatus according to a tenth aspect of the disclosure, in any one of the first to eighth aspects, the light-receiving element may be a photodiode.

In a biometric information acquiring apparatus according to an eleventh aspect of the disclosure, in any one of the first to tenth aspects, the analysis device may be configured to acquire a volume pulse wave as the biometric information.

### Supplementary Information

An aspect of the disclosure is not limited to the embodiments described above, various changes can be made within the scope of the claims, and embodiments obtained by appropriately combining the technical elements respectively disclosed in the different embodiments are also included in the technical scope of an aspect of the disclosures. Furthermore, technical elements disclosed in the respective embodiments may be combined to provide a new technical feature.

While there have been described what are at present considered to be certain embodiments of the invention, it will be understood that various modifications may be made thereto, and it is intended that the appended claim cover all such modifications as fall within the true spirit and scope of the invention.

1. A biometric information acquiring apparatus that acquires biometric information of a living body, the biometric information acquiring apparatus comprising:
a light-receiving element configured to receive light arriving from the living body and generate a light reception signal corresponding to the light; and
an analysis device configured to analyze the light reception signal to acquire the biometric information,
wherein the light-receiving element includes a first light-receiving element configured to receive first light having an intensity peak in a first wavelength band of 620 nm or more and 740 nm or less, and
the light-receiving element further includes at least one of
(i) a second light-receiving element configured to receive second light having an intensity peak in a second wavelength band of 370 nm or more and 600 nm or less, or
(ii) a third light-receiving element configured to receive third light having an intensity peak in a third wavelength band of 760 nm or more and 1100 nm or less.

2. The biometric information acquiring apparatus according to claim 1,
wherein the light-receiving element includes both the second light-receiving element and the third light-receiving element.

3. The biometric information acquiring apparatus according to claim 2,
wherein the second light-receiving element includes at least one of
(i) a first type of second light-receiving element configured to receive a first type of second light having an intensity peak in a first type of second wavelength band of 370 nm or more and 490 nm or less, or
(ii) a second type of second light-receiving element configured to receive a second type of second light having an intensity peak in a second type of second wavelength band of more than 490 nm and 590 nm or less.

4. The biometric information acquiring apparatus according to claim 3,
wherein the first light is red light,
the first type of second light is blue light,
the second type of second light is green light, and
the third light is infrared light.

5. The biometric information acquiring apparatus according to claim 1, further comprising
a spectral filter including a first filter configured to transmit the first light,
wherein the spectral filter further includes at least one of or
(i) a second filter configured to transmit the second light,
(ii) a third filter configured to transmit the third light.

6. The biometric information acquiring apparatus according to claim 1, further comprising
a light-emitting device configured to emit test light toward the living body,
wherein the light-receiving element receives the test light reflected by the living body.

7. The biometric information acquiring apparatus according to claim 6,
wherein the light-emitting device includes a first light source configured to emit the first light toward the living body as the test light, and
the light-emitting device further includes at least one of
(i) a second light source configured to emit the second light toward the living body as the test light, or
(ii) a third light source configured to emit the third light toward the living body as the test light.

8. The biometric information acquiring apparatus according to claim 6,
wherein the light-emitting device includes a white light source configured to emit white light toward the living body as the test light.

9. The biometric information acquiring apparatus according to claim 1,
wherein the light-receiving element is a solid-state imaging element.

10. The biometric information acquiring apparatus according to claim 1,
wherein the light-receiving element is a photodiode.

11. The biometric information acquiring apparatus according to claim 1,
wherein the analysis device acquires a volume pulse wave as the biometric information.
